(19) United States
(12) Patent Application Publication
(10) Pub. No.: US 2025/0214918 A1
(43) Pub. Date: Jul. 3, 2025

(54) FUNCTIONALIZATION OF TERPENES BY SELECTIVE DEPROTONATION FOR THE SYNTHESIS OF (+)-ARTEMISININ AND RELATED COMPOUNDS

(71) Applicant: BOARD OF REGENTS, THE UNIVERSITY OF TEXAS SYSTEM, Austin, TX (US)

(72) Inventors: Doug E. FRANTZ, San Antonio, TX (US); Shawn BLUMBERG, San Antonio, TX (US); Eliezer ORTIZ, San Antonio, TX (US); Nicholas CLANTON, San Antonio, TX (US); Nick WILSON, San Antonio, TX (US)

(21) Appl. No.: 18/867,712

(22) PCT Filed: May 22, 2023
(86) PCT No.: PCT/US2023/067307

§ 371 Date: Nov. 20, 2024

Related U.S. Application Data
(60) Provisional application No. 63/344,514, filed on May 20, 2022.

(51) Int. Cl. C07C 29/48; C07C 45/29; C07C 51/285

(52) U.S. Cl. CPC C07C 29/48; C07C 45/29; C07C 51/285; C07C 2602/28

(57) ABSTRACT

The present invention provides methods for the preparation of (+)-Artemisinin and intermediates thereof, including via the regioselective deprotonation of amorphadiene (AD). In other aspects, there are provided methods for the functionalization terpenes by selective deprotonation, including the utilization of 4,11-amorphadiene as an industrially viable feedstock for the synthesis of artemisinin. In some embodiments, these methods are broadly applicable to the derivatization of a variety of cyclic and linear terpenes.

This application claims the benefit of priority to U.S. Provisional Application No. 63/344,514, filed on May 20, 2022, the entire contents of which are hereby incorporated by reference.

### BACKGROUND OF THE INVENTION

### I. Field of the Invention

The present invention relates generally to the field of chemistry. More particularly, it relates to synthetic methods and intermediates for the synthesis of pharmaceuticals, including (+)-Artemisinin.

### II. Description of Related Art

Terpenes and terpenoid compounds occupy a unique niche in the world of natural products. While this term “natural product” often evokes images of complex syntheses and laborious isolations, the terpenes stand in contrast as some of the most industrially significant chemical feedstocks. The case of isolation of hydrocarbon terpenes from biological sources makes them excellent candidates for the replacement of petroleum-derived compounds. While several of these compounds are already in-use in large-scale chemical processes, the push to transition to biorenewable feedstocks will increase the demand for highly selective and robust transformations on this class of compounds.

The utilization of terpene biosynthetic precursors provides an opportunity to develop a new route to pharmaceuticals, including, for example, the antimalarial drug artemisinin. This compound is the principle active component from the sweet wormwood plant (Artemisia annua) used for centuries in traditional Chinese medicine.

The latest World Malaria Report from the World Health Organization (WHO) estimated 241 million cases of malaria and 627,000 deaths in 2020. (World Malaria Report, 2021) Unfortunately, these numbers represent a significant increase compared to 2019 and were exacerbated by disruptions in prevention, diagnosis, and treatment of malaria due to the ongoing COVID-19 pandemic. Further disruptions in global supplies chains continue to negatively impact the production of antimalarials and ultimately contribute to reduced gains in malaria-endemic countries. (Hampshire, 2021; Socal et al., 2021; Hussein et al., 2020; Heuschen et al., 2021) As a result, the WHO has recently warned that the convergence of multiple threats could thwart efforts to reach global malaria targets in the foreseeable future.

In the battle against malaria, artemisinin-based combined therapies (ACTs) remain as the first-line arsenal for the treatment of uncomplicated disease caused by P. falciparum and in select cases caused by P. vivax. (Pousibet-Puerto et al., 2016) Global demand for ACTs continues to grow, up to 218 metric tons in 2021, (Global Malaria Diagnostic and Artemisinin Treatment Commodities Demand Forecast 2017-2021, 2022) however meeting this demand has been hampered not only due to COVID-19 but the long-term supply issues of artemisinin itself. During the past 20 years, the world's supply and cost of artemisinin has been notably erratic. Extraction from Artemisia annua L. continues to be the major source of this API (100-120 tons/year), however, sustainable supplies are dependent on varying market dynamics, climate change, geographical location, and geopolitical pressures. (Shretta and Yadav, 2012; White, 2008) The remaining gap in artemisinin stock is supplemented by semisynthetic industrial approaches (50-60 tons/year) that rely on the biosynthetic production of artemisinic acid (AA) via fermentation of sugar in titers of ˜25 g/L using genetically engineered strains of Saccharomyces cerevisiae (brewer's yeast). (Paddon et al., 2013; Kung et al., 2018; Ro et al. m 2006) Significant effort has since been put forth to optimize the chemical transformation of AA to artemisinin. (Fan et al., 2012; Feth et al., 2013; Horvath et al., 2015; Levesque et al., 2012; Lee et al., 2017; Amara et al., 2015) Despite these elegant strategies, the cost of semisynthetic approaches (350-400 $/kg) still do not compete with the current price of artemisinin obtained by extraction (250 $/kg).

Interestingly, the initial fermentation step used to produce amorph-4,11-diene (amorphadiene, AD) is capable of producing titers that are up to 5 times higher (˜120 g/L) than was obtainable for AA. (Westfall et al, 2012; Martin, 2003) Thus, realizing that AD could serve as a more attractive precursor to develop a semisynthetic route to artemisinin, Amyris developed two approaches to oxidize AD via either selective hydroboration/oxidation or epoxidation of the exocyclic double bond. However, both of these routes were abandoned and deemed too costly at the time. (Reiling et al., 2006) Renewed efforts to utilize AD involved a 6-step synthetic sequence beginning with selective epoxidation of the endocyclic double bond allowing for subsequent manipulation of the exocyclic olefin that ultimately culminated with a Li-metal mediated reductive removal of the epoxide to provide dihydroartemisinic acid (DHAA). (Singh et al., 2017) However, whether this route can be scaled up to production scale remains to be determined.

Biomimetic approaches that target intermediates along biosynthetic pathways are proven strategies for the total synthesis of natural products. (Chen et al., 2009; Harmange Magnani, 2020; Hung et al., 2019; Bao et al., 2021) From this perspective, the development of a direct chemical conversion of AD to artemisinic alcohol, the next intermediate on the biosynthetic pathway to artemisinin, could provide advantages over previous semisynthetic approaches. In turn, this strategy would complement existing total syntheses of artemisinin that start from commodity raw materials, (Avery et al., 1987; Liu et al., 1996; Avery et al., 2002; Ravindranathan et al., 1990; Constantino et al., 1996; Tang et al., 2018) most recent of these being the approach developed by Cook. (Zhu et al., 2012)

While highly effective, the use of artemisinin is impacted by a volatile supply chain that relies heavily on direct isolation of the product from cultivated plants. This fluctuation in supply leads to unstable pricing. Multiple reliable feedstocks for artemisinin production are needed in order to stabilize the supply, and therefore the cost. Therefore, more practical methods for the synthesis of (+)-Artemisinin would be a great advantage.

### SUMMARY OF THE INVENTION

In one aspect, the present disclosure provides methods for the preparation of (+)-Artemisinin, intermediates thereof, and related compounds. In some embodiments, the methods are those in the claims section, the illustrative embodiments, and the examples, which are incorporated herein by reference.

Other objects, features and advantages of the present disclosure will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present disclosure. The invention may be better understood by reference to one of these drawings in combination with the detailed description of specific embodiments presented herein.

FIGS. 1a-c—(a) Engineered Biosynthesis Towards the Industrial Semisynthesis of (+)-Artemisinin; (b) Pd-Catalyzed Oxidation of Amorphadiene (AD) by Cossy and Amara; (c) Regioselective Deprotonation/Oxidation of AD.

FIG. 2—This synthetic scheme illustrates the through process for the conversion of AD to AA. Regioselective deprotonation/oxidation of AD using KTMP provided artemisinic alcohol 3 after borylation/oxidation in 89% assay yield as determined by qNMR analysis. Oxidation of crude 3 to artemisinic aldehyde 4 was realized via a Cu-catalyzed oxidation using O₂ as the stoichiometric oxidant in 92% HPLC assay yield of 4. Subsequent conversion of 4 to 5 was achieved in 98% HPLC assay yield after 24 h via a Pinnick oxidation using 2-methyl-2-butene (15 equiv) as the scavenger for the HOCl byproduct. Isolation via direct crystallization from this crude reaction mixture provided artemisinic acid 5 in 53% overall yield from AD resulting in an average yield of 81% for each of the three steps.

FIG. 3—This table provides the results for the deprotonation on a broad range of terpenes. 1.5 equivalents of base was chosen for the prototypical reaction conditions.

### DESCRIPTION OF ILLUSTRATIVE EMBODIMENTS

In one aspect of the present disclosure, there are provided methods for the preparation of (+)-Artemisinin and intermediates thereof, including via the regioselective deprotonation of amorphadiene (AD). In other aspects, there are provided methods for the functionalization terpenes by selective deprotonation, including the utilization of 4,11-amorphadiene as an industrially viable feedstock for the synthesis of artemisinin. In some embodiments, these methods are broadly applicable to the derivatization of a variety of cyclic and linear terpenes. Additional details are provided below.

### I. Functionalization Terpenes by Selective Deprotonation

Direct chemo- and site-selective C—H functionalization of complex naturally occurring terpenes, such as AD, remains a formidable challenge for synthetic organic chemists. There are over 30,000 known terpenes that could serve as biorenewable hydrocarbon feedstocks, yet the laboratory toolbox still lacks a crescent wrench to adjust our methods to each and every synthetic bolt (terpene in this case) that needs tightening. (Chen et al., 2009; Abrams et al., 2018; Kanda et al., 2020; Teh et al., 2020) From this perspective, in an age where catalytic C—H functionalization are making great strides, simple and efficient stoichiometric functionalizations can sometimes be overlooked. (Hughes et al., 2018) For example, regioselective stoichiometric deprotonations have been a proven strategy for the functionalization of unsaturated hydrocarbons, including terpenes, since the 1970s. The landmark study by Crawford on the direct metalation of limonene using n-BuLi-TMEDA catalyzed a series subsequent investigations by others in the field. (Crawford et al., 1972) In the 1980's Schlosser's “superbase” system (Schlosser, 1988; Schlosser, 1984) that combined n-BuLi with KO′Bu (LICKOR) gained notoriety and was demonstrated in the selective allylic deprotonation of simple olefins and terpenoids. (Schlosser, 1999) However, the application of these deprotonation strategies to more complex sesquiterpenes, including AD, has yet to be demonstrated systematically.

In one aspect of the present disclosure, there are provided methods for the preparation of (+)-Artemisinin, including via the regioselective deprotonation of amorphadiene (AD). Also provided are methods and for the functionalization terpenes by selective deprotonation, including the utilization of 4,11-amorphadiene as an industrially viable feedstock for the synthesis of artemisinin. In some embodiments, these methods are broadly applicable to a variety of cyclic and linear terpenes, including those described in Example 3 below. In some embodiments there are provided methods for the direct allylic C—H functionalization of amorphadiene (AD) to artemisinic alcohol via a highly regioselective deprotonation. In some of these methods, KTMP is used as a base. In some of the embodiments, KTMP demonstrates superior regioselectivity for deprotonation at C12 over 4 other possible allylic sites in AD.

In some embodiments, these methods can be extrapolated, thereby, for example, providing the first telescoped chemical synthesis of artemisinic acid (AA) from AD. In some embodiments, there are provided methods for large-scale semisynthetic production of artemisinin. In other embodiments, there are provided methods additional C—H functionalization of AD using various electrophiles is also provided.

These methods can be further modified and optimized using the principles and techniques of organic chemistry as applied by a person skilled in the art. Such principles and techniques are taught, for example, in March's Advanced Organic Chemistry: Reactions, Mechanisms, and Structure (2013), which is incorporated by reference herein.

### II. Methods and Intermediates for the Synthesis of (+)-Artemisinin

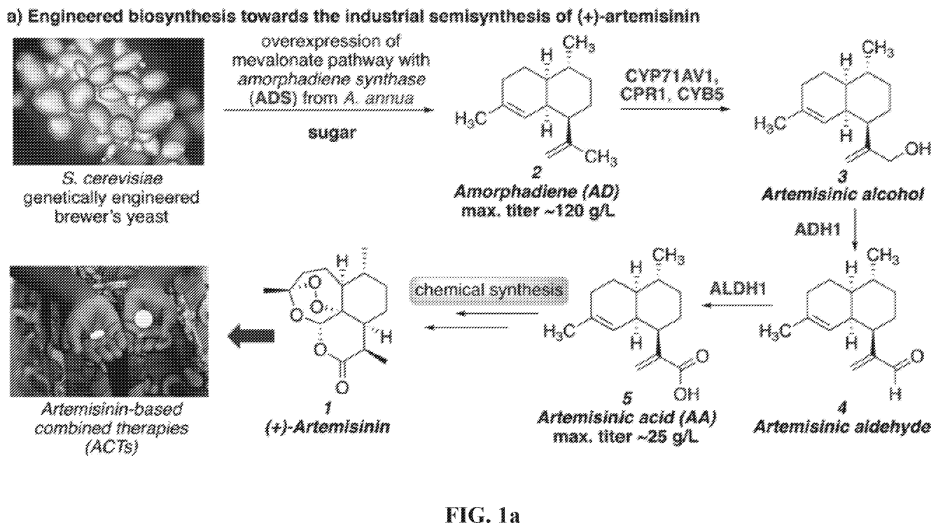

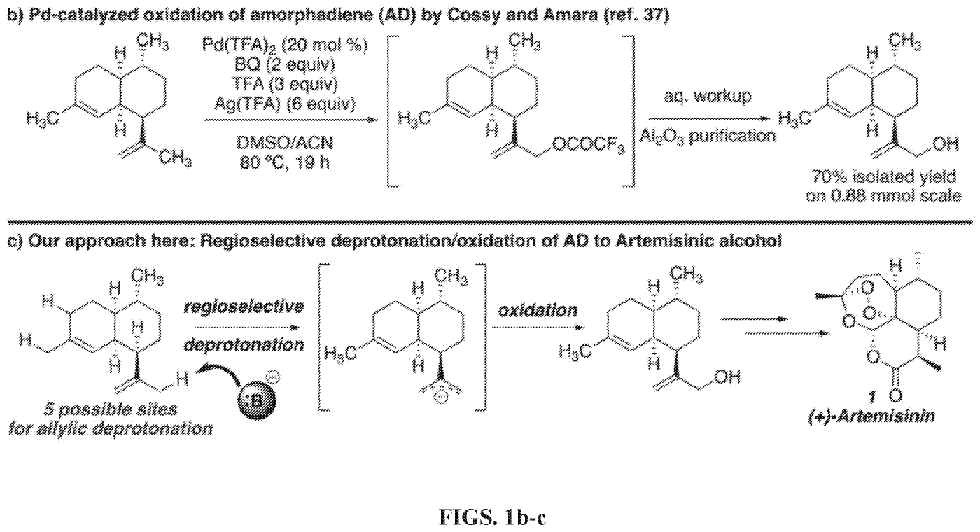

In another aspect, there are provided improved methods for making (+)-Artemisinin. Biomimetic approaches that target intermediates along biosynthetic pathways are proven strategies for the total synthesis of natural products. (Chen et al., 2009; Hamange Magnani et al., 2020; Hung et al., 2019; Bao et al., 2021) From this perspective, the development of a direct chemical conversion of AD to artemisinic alcohol, the next intermediate on the biosynthetic pathway to artemisinin, provide significant advantages over previous semisynthetic approaches. In turn, the methods provided herein complement existing total syntheses of artemisinin that start from commodity raw materials, (Avery et al., 1987; Liu et al., 1996; Avery et al., 2002; Ravindranathan et al., 1990; Constantino et al., 1996; Tang et al., 2018) most recent of these being the graceful approach developed by Cook. (Zhu et al., 2012) The Cossy and Amara groups demonstrated various methods for the functionalization of AD, (Gomez Fernandez et al., 2021; Zanetti et al., 2020; Schwertz et al., 2020) in particular the Pd-catalyzed regioselective oxidation of AD. (Zanetti et al., 2021) In some embodiments, there is provided a robust and efficient approach for the direct conversion of AD to artemisinic alcohol as a biomimetic formal total synthesis to artemisinin. See FIG. 1 and the examples below.

These methods can be further modified and optimized using the principles and techniques of organic chemistry as applied by a person skilled in the art. Such principles and techniques are taught, for example, in March's Advanced Organic Chemistry: Reactions, Mechanisms, and Structure (2013), which is incorporated by reference herein.

### III. Process Scale-Up

The above methods can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein.

### IV. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof, in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” represents a single bond, “═” represents a double bond; and “≡” represents triple bond. The symbol “ [symbol] ” represents an optional bond, which if present is either single or double. Unless indicated otherwise, the symbol “ [symbol] ” represents a single bond or a double bond. Furthermore, it is noted that the single bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

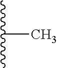

for methyl) indicates a point of attachment of the group. It is noted that the point of attachment is typically only identified in this manner for larger groups in order to assist the reader in unambiguously identifying a point of attachment. The symbol “ [symbol] ” represents a single bond where the group attached to the thick end of the wedge is “out of the page.” The symbol “ [symbol] ” represents a single bond where the group attached to the thick end of the wedge is “into the page”. The symbol “ [symbol] ” represents a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C₁₋₄-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic 71 system. An aromatic compound or chemical group may be depicted as a single resonance structure; however, depiction of one resonance structure is taken to also refer to any other resonance structure. For example:

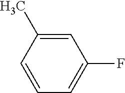

is also taken to refer to

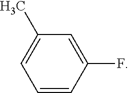

Aromatic compounds may also be depicted using a circle to represent the delocalized nature of the electrons in the fully conjugated cyclic π system, two non-limiting examples of which are shown below:

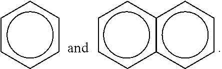

The term “alkyl” refers to a monovalent saturated aliphatic group with a carbon atom as the point of attachment, a linear or branched acyclic structure, and no atoms other than carbon and hydrogen. The groups —CH₃(Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, t-butyl, t-Bu or ^{t}Bu), and —CH₂C(CH₃)₃(neo-pentyl) are non-limiting examples of alkyl groups. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂(cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

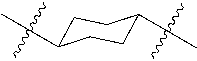 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “cycloalkenyl” refers to a monovalent saturated aliphatic group with a carbon atom as the point of attachment, said carbon atom forming part of one or more non-aromatic ring structures, at least one nonaromatic carbon-carbon double bond, no carbon-carbon triple bonds, and no atoms other than carbon and hydrogen. Non-limiting examples include: —CH(CH)₂ (cyclopropenyl), cyclobutenyl, cyclopentenyl, or cyclohexenyl. As used herein, the term does not preclude the presence of one or more alkyl groups (carbon number limitation permitting) attached to a carbon atom of the non-aromatic ring structure.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present.

Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). An “arene” refers to the class of compounds having the formula H—R, wherein R is aryl as that term is defined above. Benzene and toluene are non-limiting examples of arenes.

Some of the abbreviations used herein are as follows: Ac indicates an acetyl group (—C(O)CH₃) Boc refers to tert-butyloxycarbonyl; CCR2 refers to CC chemokine receptor 2; CCL2 refers to CC chemokine ligand 2; CCR5 refers to CC chemokine receptor 5; DBDMH refers to 1,3-Dibromo-5,5-dimethylhydantoin; DIBAL-H is diisobutylaluminium hydride; DMAP refers to 4-dimethylaminopyridine; DMF is dimethylformamide; DMSO is dimethyl sulfoxide; EDC refers to 1-ethyl-3-(3-dimethylaminopropyl)carbodiimide; Et₂O, diethyl ether; KTMP refers to potassium 2,2,6,6-tetramethylpiperidine; NCS refers to N-Chlorosuccinimide; NMO refers to N-methylmorpholine N-oxide; Py stands for Pyridine; T3P refers to propylphosphonic anhydride; TFA is trifluoroacetic acid; THE is tetrahydrofuran; TLC refers to thin layer chromatography; TNFα or TNF-α refer to tumor necrosis factor-α; TPAP is tetrapropylammonium perruthenate; Ts stands for tosyl; TsOH or p-TsOH is p-toluenesulfonic acid.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “isomer” of a first compound is a separate compound in which each molecule contains the same constituent atoms as the first compound, but where the configuration of those atoms in three dimensions differs.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer.

In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The above definitions supersede any conflicting definition in any of the reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Selective Deprotonation of AD

A selected subset of our initial exploratory reactions for the selective deprotonation of AD followed by borylation/oxidation is presented in Table 1 below. Preliminary proof of concept was realized using n-BuLi-TMEDA in hexanes; conditions previously shown to metalate limonene. (Crawford et al., 1972) However, conversion of AD to 3 was low with concomitant formation of regioisomeric allylic alcohol 6 (Table 1, entries 1 and 3). Even lower conversion was realized using a s-BuLi-TMEDA combination (Table 1, entry 2). Significant conversion was not realized until the ternary combination of n-BuLi, KO^{t}Bu, and 2,2,6,6-tetramethylpiperidine (TMP) in THE was employed (Table 1, entry 7). Additional optimization of this result using 2 equivalents of each reagent provided high conversion of AD (89%), exceptional regioselectivity (>20:1), and an excellent isolated yield of 3 (81%) (Table 1, entry 9).

TABLE 1
Initial exploration of the regioselective deprotonation of AD.

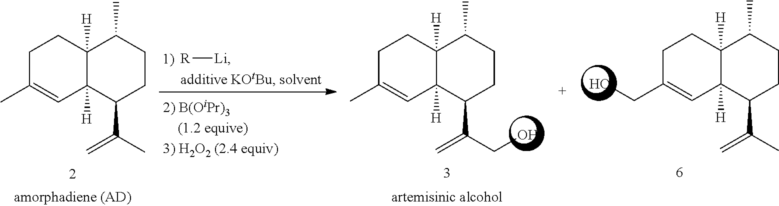

| | R-Li | additive | | | | | conversion of AD | | |
|---|---|---|---|---|---|---|---|---|---|
| entry^{a} | (equiv) | (equiv) | KO^{t}Bu (equiv) | solvent | temp (° C.) | time (h) | (%)^{b} | yield (%)^{c} | 3:6^{d} |
| 1 | n-BuLi | TMEDA | none | hexanes | 0 | 16 | 35 | ND | 3:1 |
| | (0.67) | (0.67) | | | | | | | |
| 2 | s-BuLi | TMEDA | none | cyclohexane | 0 | 16 | 6 | ND | 5:1 |
| | (0.67) | (0.67) | | | | | | | |
| 3 | n-BuLi | none | 1.0 | hexanes | 0 | 24 | 25 | ND | 2:1 |
| | (1.0) | | | | | | | | |
| 4 | n-BuLi | none | none | THF | −78 | 1 | <5 | ND | ND |
| | (1.0) | | | | | | | | |
| 5 | n-BuLi | none | 1.2 | THF | −78 | 1 | <5 | ND | ND |
| | (1.2) | | | | | | | | |
| 6 | n-BuLi | 1.2 | none | THF | −78 | 1 | <5 | ND | ND |
| | (1.2) | | | | | | | | |
| 7 | n-BuLi | TMP | 1.2 | THF | −78 | 1 | 72 | 60 | >50:1 |
| | (1.2) | (1.2) | | | | | | | |
| 8 | n-BuLi | TMP | 1.5 | THF | −78 | 1 | 78 | 72 | >50:1 |
| | (1.5) | (1.5) | | | | | | | |
| 9 | n-BuLi | TMP | 2.0 | THF | −78 | 1 | 89 | 81 | >50:1 |
| | (2.0) | (2.0) | | | | | | | |
| 10 | n-BuLi | TMP | 3.0 | heptane | 23 | 24 | 68 | ND | 3:1 |
| | (3.0) | (3.0) | | | | | | | |

^{a}All reactions performed on a 0.5 mmol scale of AD.
^{b}Conversions determined via ¹H NMR analysis of crude reaction mixtures by comparing the relative amounts of AMD to 3 and 6 combined.
^{c}Isolated yields.
^{d}Ratios determined on crude reaction mixtures by ¹H NMR.

Reactions performed without TMP (Schlosser's LICKOR conditions), without KO^{t}Bu (generating LiTMP), or just n-BuLi alone failed to provide any measurable conversion by ¹H NMR (Table 1, entries 4-6). These data taken together provide supporting evidence that, at least in some embodiments, KTMP (potassium 2,2,6,6-tetramethylpiperidine) is required to achieve high conversion and regioselectivity for the deprotonation of AD.

Also explored were reaction conditions using KTMP in hydrocarbon solvents where increase thermal stability is known over ethereal solvents. (Armstrong et al., 2008) Conversion (68%) and regioselectivity (3:1) was realized using heptane as the solvent at room temperature with 3 equivalents of KTMP (Table 1, entry 10).

### Example 2—Conversion of AD to Artemisinic Acid

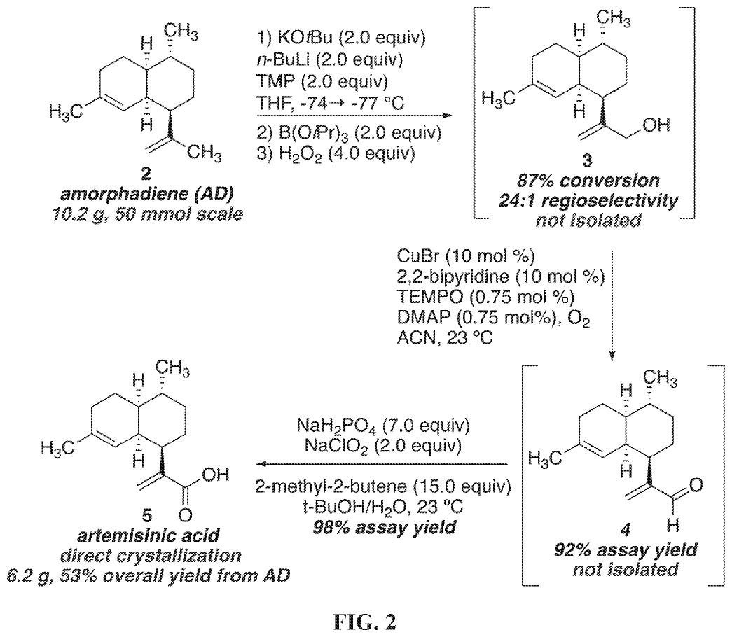

With a highly regioselective and robust deprotonation/oxidation of AD in hand, attention was directed to obtaining preliminary proof-of-concept that a through process to convert AD to artemisinic acid (5) without isolation or purification of any intermediates was feasible. A two-step/one-pot oxidation sequence was identified using crude artemisinic alcohol (3) obtained from the regioselective deprotonation to provide 5 in high isolated yield on a 50 mmol scale (FIG. 2). Regioselective deprotonation/oxidation of AD using KTMP as described above provided artemisinic alcohol 3 after borylation/oxidation in 89% assay yield as determined by qNMR analysis. Oxidation of crude 3 to artemisinic aldehyde 4 was realized via a Cu-catalyzed oxidation using O₂ as the stoichiometric oxidant in 92% HPLC assay yield of 4. Subsequent conversion of 4 to 5 was achieved in 98% HPLC assay yield after 24 h via a Pinnick oxidation using 2-methyl-2-butene (15 equiv) as the scavenger for the HOCl byproduct. Isolation via direct crystallization from this crude reaction mixture provided artemisinic acid 5 in 53% overall yield from AD resulting in an average yield of 81% for each of the three steps.

### Example 3—Conversion of AD to Artemisinic Acid

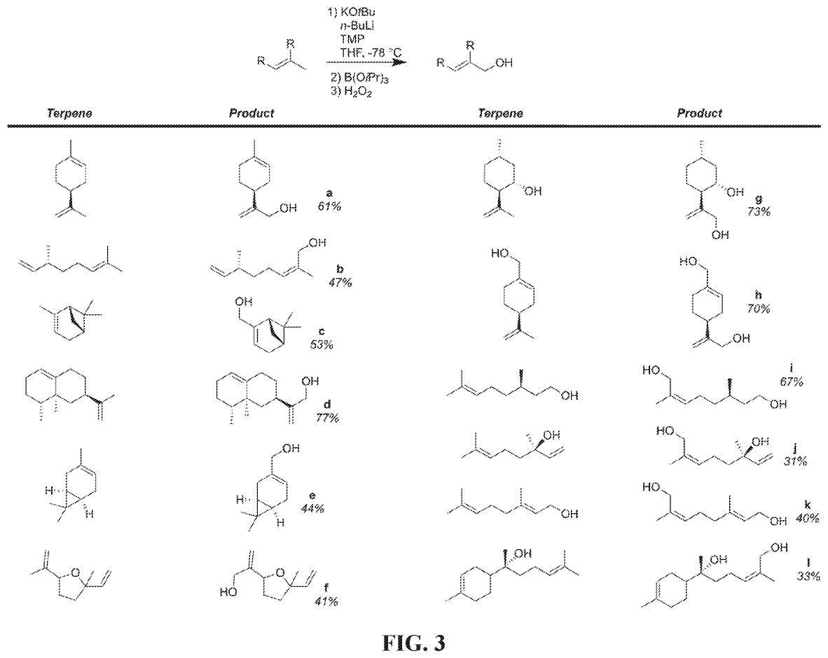

A broad range of terpenes were examined. 1.5 equivalents of base was chosen for the prototypical reaction conditions, as increasing further showed little impact on isolated yield. The results of this deprotonation on a broad range of terpenes are shown in the table of FIG. 3. First, it should be noted that terpenols are also well-tolerated with the addition of an extra equivalent of base. Many of these compounds, (as in the case of limonene and perilyl alcohol) perform comparably to their hydrocarbon counterparts. Both cyclic and linear terpenes perform well with excellent selectivity. Of note, the deprotonation of valencene was accomplished using low-purity (about 74%) food grade material. The product alcohol was isolated in >98% purity with no other observable terpenoid products. Thus, this represents an excellent method for the transformation of low-grade feedstocks into higher value products. The deprotonation of limonene was performed on 5 g scale (36 mmol) with no decrease in yield.

All of the methods and compositions disclosed and claimed herein can be made and executed without undue experimentation in light of the present disclosure. While the methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references to the extent that they provide exemplary procedural or other details supplementary to those set forth herein, are specifically incorporated herein by reference.
Anderson, Practical Process Research & Development, 2000.
Smith, March's Advanced Organic Chemistry: Reactions, Mechanisms, and Structure, 7^{th} Ed., Wiley, 2013.
World Malaria Report. https://www.who.int/teams/global-malaria-programme/reports/world-malaria-report-2021 (accessed Feb. 4, 2022).
Hampshire, K.; David-Barrett, L.; Osman, Mariwah, S.; Hamill, H., The impact of COVID-19 on antimalarial supply chains in Ghana. The Lancet Global Health 2021, 9 (9).
Socal, M. P.; Sharfstein, J. M.; Greene, J. A., The Pandemic and the Supply Chain: Gaps in Pharmaceutical Production and Distribution. Am J Public Health 2021, 111 (4), 635-639.
Hussein, M. I. H.; Albashir, A. A. D.; Elawad, O.; Homeida, A., Malaria and COVID-19: unmasking their ties. Malar J 2020, 19 (1), 457.
Heuschen, A. K.; Lu, G.; Razum, O.; Abdul-Mumin, A.; Sankoh, O.; von Seidlein, L.; D'Alessandro, U.; Muller, O., Public health-relevant consequences of the COVID-19 pandemic on malaria in sub-Saharan Africa: a scoping review. Malar J 2021, 20 (1), 339.
Pousibet-Puerto, J.; Salas-Coronas, J.; Sanchez-Crespo, A.; Molina-Arrebola, M. A.; Soriano-Perez, M. J.; Gimenez-Lopez, M. J.; Vazquez-Villegas, J.; Cabezas-Fernandez, M. T., Impact of using artemisinin-based combination therapy (ACT) in the treatment of uncomplicated malaria from Plasmodium falciparum in a non-endemic zone. Malar J 2016, 15 (1), 339.
Global Malaria Diagnostic and Artemisinin Treatment Commodities Demand Forecat 2017-2021. https://unitaid.org/assets/Global-malaria-diagnostic-and-artemisinin-treatment-commodities-demand-forecast-2017-2021-Report-May-2018.pdf (accessed Feb. 3, 2022).
Shretta, R.; Yadav, P., Stabilizing supply of artemisinin and artemisinin-based combination therapy in an era of wide-spread scale-up. Malar J 2012, 11, 399.
White, N. J., Qinghaosu (artemisinin): the price of success. Science 2008, 320 (5874), 330-4.
Paddon, C. J.; Westfall, P. J.; Pitera, D. J.; Benjamin, K.; Fisher, K.; McPhee, D.; Leavell, M. D.; Tai, A.; Main, A.; Eng, D.; Polichuk, D. R.; Teoh, K. H.; Reed, D. W.; Treynor, T.; Lenihan, J.; Fleck, M.; Bajad, S.; Dang, G.; Dengrove, D.; Diola, D.; Dorin, G.; Ellens, K. W.; Fickes, S.; Galazzo, J.; Gaucher, S. P.; Geistlinger, T.; Henry, R.; Hepp, M.; Homing, T.; Iqbal, T.; Jiang, H.; Kizer, L.; Lieu, B.; Melis, D.; Moss, N.; Regentin, R.; Secrest, S.; Tsuruta, H.; Vazquez, R.; Westblade, L. F.; Xu, L.; Yu, M.; Zhang, Y.; Zhao, L.; Lievense, J.; Covello, P. S.; Keasling, J. D.; Reiling, K. K.; Renninger, N. S.; Newman, J. D., High-level semi-synthetic production of the potent antimalarial artemisinin. Nature 2013, 496 (7446), 528-32.
Kung, S. H.; Lund, S.; Murarka, A.; McPhee, D.; Paddon, C. J., Approaches and Recent Developments for the Commercial Production of Semi-synthetic Artemisinin. Front Plant Sci 2018, 9, 87.
Ro, D. K.; Paradise, E. M.; Ouellet, M.; Fisher, K. J.; Newman, K. L.; Ndungu, J. M.; Ho, K. A.; Eachus, R. A.; Ham, T. S.; Kirby, J.; Chang, M. C.; Withers, S. T.; Shiba, Y.; Sarpong, R.; Keasling, J. D., Production of the antimalarial drug precursor artemisinic acid in engineered yeast. Nature 2006, 440 (7086), 940-3.
Fan, X.; Sans, V.; Yaseneva, P.; Plaza, D. D.; Williams, J.; Lapkin, A., Facile Stoichiometric Reductions in Flow: An Example of Artemisinin. Organic Process Research & Development 2012, 16 (5), 1039-1042.
Feth, M. P.; Rossen, K.; Burgard, A., Pilot Plant PAT Approach for the Diastereoselective Diimide Reduction of Artemisinic Acid. Organic Process Research & Development 2013, 17 (2), 282-293.
Horváth, Z.; Horosanskaia, E.; Lee, J. W.; Lorenz, H.; Gilmore, K.; Seeberger, P. H.; Seidel-Morgenstern, A., Recovery of Artemisinin from a Complex Reaction Mixture Using Continuous Chromatography and Crystallization. Organic Process Research & Development 2015, 19 (6), 624-634.
Levesque, F.; Seeberger, P. H., Continuous-flow synthesis of the anti-malaria drug artemisinin. Angew Chem Int Ed Engl 2012, 51 (7), 1706-9.
Lee, D. S.; Amara, Z.; Clark, C. A.; Xu, Z.; Kakimpa, B.; Morvan, H. P.; Pickering, S. J.; Poliakoff, M.; George, M. W., Continuous Photo-Oxidation in a Vortex Reactor: Efficient Operations Using Air Drawn from the Laboratory. Org Process Res Dev 2017, 21 (7), 1042-1050.
Amara, Z.; Bellamy, J. F.; Horvath, R.; Miller, S. J.; Beeby, A.; Burgard, A.; Rossen, K.; Poliakoff, M.; George, M. W., Applying green chemistry to the photochemical route to artemisinin. Nat Chem 2015, 7 (6), 489-95.
Westfall, P. J.; Pitera, D. J.; Lenihan, J. R.; Eng, D.; Woolard, F. X.; Regentin, R.; Horning, T.; Tsuruta, H.; Melis, D. J.; Owens, A.; Fickes, S.; Diola, D.; Benjamin, K. R.; Keasling, J. D.; Leavell, M. D.; McPhee, D. J.; Renninger, N. S.; Newman, J. D.; Paddon, C. J., Production of amorphadiene in yeast, and its conversion to dihydroartemisinic acid, precursor to the antimalarial agent artemisinin. Proc Natl Acad Sci USA 2012, 109 (3), E111-8.
Martin, V. J.; Pitera, D. J.; Withers, S. T.; Newman, J. D.; Keasling, J. D., Engineering a mevalonate pathway in Escherichia coli for production of terpenoids. Nat Biotechnol 2003, 21 (7), 796-802.
Reiling, K. K. R., N. S.; McPhee, D. J.; Fisher, K. J.; Ockey, D. A. Conversion of amorpha-4,11,-diene to artemisinin and artemisinin precursors. 2006.
Singh, D.; McPhee, D.; Paddon, C. J.; Cherry, J.; Maurya, G.; Mahale, G.; Patel, Y.; Kumar, N.; Singh, S.; Sharma, B.; Kushwaha, L.; Singh, S.; Kumar, A., Amalgamation of Synthetic Biology and Chemistry for High-Throughput Nonconventional Synthesis of the Antimalarial Drug Artemisinin. Organic Process Research & Development 2017, 21 (4), 551-558.
Chen, K.; Baran, P. S., Total synthesis of eudesmane terpenes by site-selective C—H oxidations. Nature 2009, 459 (7248), 824-8.
Harmange Magnani, C. S.; Thach, D. Q.; Haelsig, K. T.; Maimone, T. J., Syntheses of Complex Terpenes from Simple Polyprenyl Precursors. Acc Chem Res 2020, 53 (4), 949-961.
Hung, K.; Condakes, M. L.; Novaes, L. F. T.; Harwood, S. J.; Morikawa, T.; Yang, Z.; Maimone, T. J., Development of a Terpene Feedstock-Based Oxidative Synthetic Approach to the Illicium Sesquiterpenes. J Am Chem Soc 2019, 141 (7), 3083-3099.
Bao, R.; Zhang, H.; Tang, Y., Biomimetic Synthesis of Natural Products: A Journey To Learn, To Mimic, and To Be Better. Acc Chem Res 2021, 54 (19), 3720-3733.
Avery, M. A.; Jennings-White, C.; Chong, W. K. M., The Total synthesis of (+)-artemisinin and (+)-9-desmethyltemesinin. Tetrahedron Letters 1987, 28 (40), 4629-4632.
Liu, H. J.; Yeh, W. L., Total Synthesis of (−)-Qinghaosu IV (Artemisinin D, Arteannuin D). Heterocycles 1996, 42 (2).
Avery, M. A.; Chong, W. K. M.; Jennings-White, C., Stereoselective total synthesis of (+)-artemisinin, the antimalarial constituent of Artemisia annua L. Journal of the American Chemical Society 2002, 114 (3), 974-979.
Ravindranathan, T.; Anil Kumar, M.; Menon, R. B.; Hiremath, S. V., Stereoselective synthesis of artemisinin. Tetrahedron Letters 1990, 31 (5), 755-758.
Constantino, M. G.; Beltrame, M.; da Silva, G. V. J.; Zukerman-Schpector, J., A Novel Asymmetric Total Synthesis of (+)-Artemisinin. Synthetic Communications 1996, 26 (2), 321-329.
Tang, X.; Demiray, M.; Wirth, T.; Allemann, R. K., Concise synthesis of artemisinin from a farnesyl diphosphate analogue. Bioorg Med Chem 2018, 26 (7), 1314-1319.
Zhu, C.; Cook, S. P., A concise synthesis of (+)-artemisinin. J Am Chem Soc 2012, 134 (33), 13577-9.
Gomez Fernandez, M. A.; Nascimento de Oliveira, M.; Zanetti, A.; Schwertz, G.; Cossy, J.; Amara, Z., Photochemical Hydrothiolation of Amorphadiene and Formal Synthesis of Artemisinin via a Pummerer Rearrangement. Org Lett 2021, 23 (15), 5593-5598.
Zanetti, A.; Chaumont-Olive, P.; Schwertz, G.; Nascimento de Oliveira, M.; Gomez Fernandez, M. A.; Amara, Z.; Cossy, J., Crystallization-Induced Diastereoisomer Transformation of Dihydroartemisinic Aldehyde with the Betti Base. Org Process Res Dev 2020, 24 (5), 850-855.
Schwertz, G.; Zanetti, A.; de Oliveira, M. N.; Fernandez, M. A. G.; Amara, Z.; Cossy, J., Chemo- and Diastereoselective Hydrosilylation of Amorphadiene toward the Synthesis of Artemisinin. J Org Chem 2020, 85 (15), 9607-9613.
Zanetti, A.; Schwertz, G.; de Oliveira, M. N.; Gomez Fernandez, M. A.; Amara, Z.; Cossy, J., Palladium-Catalyzed Regioselective Allylic Oxidation of Amorphadiene, a Precursor of Artemisinin. J Org Chem 2021, 86 (11), 7603-7608.
Abrams, D. J.; Provencher, P. A.; Sorensen, E. J., Recent applications of C—H functionalization in complex natural product synthesis. Chemical Society Reviews 2018, 47 (23), 8925-8967.
Kanda, Y.; Ishihara, Y.; Wilde, N. C.; Baran, P. S., Two-Phase Total Synthesis of Taxanes: Tactics and Strategies. J Org Chem 2020, 85 (16), 10293-10320.
Teh, W. P.; Obenschain, D. C.; Black, B. M.; Michael, F. E., Catalytic Metal-free Allylic C—H Amination of Terpenoids. J Am Chem Soc 2020, 142 (39), 16716-16722.
Hughes, J. M. E.; Gleason, J. L., A bio-inspired cascade and a late-stage directed sp3 C H lithiation enables a concise total synthesis of (−)-virosaine A. Tetrahedron 2018, 74 (8), 759-768.
Crawford, R. J.; Erman, W. F.; Broaddus, C. D., Metalation of limonene. Novel method for the synthesis of bisabolane sesquiterpenes. J. Am. Chem. Soc. 1972, 94 (12), 4298-4306.
Schlosser, M., Superbases for organic synthesis. Pure Appl. Chem. 1988, 60 (11), 1627-1634.
Schlosser, M.; Strunk, S., The “super-basic” butyllithium/potassium tert-butoxide mixture and other lickor-reagents. Tetrahedron Lett. 1984, 25 (7), 741-744.
Schlosser, M.; Kotthaus, M., Isopulegol as a Model Compound: Metalation and Substitution of an Allylic Position in the Presence of an Unprotected Hydroxy Function. European Journal of Organic Chemistry 1999, 1999 (2), 459-462.
Armstrong, D. R.; Graham, D. V.; Kennedy, A. R.; Mulvey, R. E.; O'Hara, C. T., A structural and computational study of synthetically important alkali-metal/tetramethylpiperidide (TMP) amine solvates. Chemistry 2008, 14 (26), 8025-34.

What is claimed is:

1. A method for the preparation of a compound of Formula II comprising deprotonating and oxidizing a compound of Formula I: 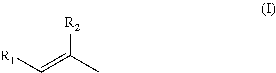
with one or more reagents under suitable conditions to make a compound of Formula II: 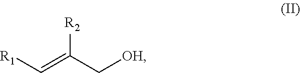
wherein:
R₁ and R₂ are each independently H, alkenyl_{(C≤12)}, substituted alkenyl_{(C≤12)}, cycloalkenyl_{(C≤12)}, or substituted cycloalkenyl, with the proviso that at least one of R₁ and R₂ is not hydrogen; or
R₁ and R₂ are taken together to form an alkenediyl_{(C≤12)} or a substituted alkenediyl_{(C≤12)}.

2. The method of claim 1, wherein the one or more reagents comprise n-BuLi, KOtBu, and 2,2,6,6-tetramethylpiperidine (TMP).

3. The method of either one of claim 1 or 2, wherein the deprotonating and oxidizing is performed in an organic solvent.

4. The method of claim 3, wherein the organic solvent is tetrahydrofuran (THF).

5. The method of any one of claims 1-4, wherein the deprotonating and oxidizing is performed at least in part at a temperature below 70° C.

6. The method of any one of claims 1-5, wherein the deprotonation occurs at a regioselectivity of approximately 3:1 or greater.

7. The method of any one of claims 1-6, wherein the oxidizing further comprises a borylation.

8. The method of any one of claims 1-7, wherein the one or more reagents comprise B(OiPr)₃ and an oxidizing agent.

9. The method of claim 9, wherein the oxidizing agent is hydrogen peroxide.

10. A method for the preparation of artemisinic alcohol comprising deprotonating and oxidizing 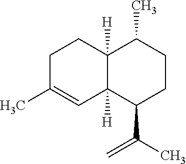
with one or more reagents under suitable conditions to make artemisinic alcohol.

11. The method of claim 11, wherein the one or more reagents comprise n-BuLi, KOtBu, and 2,2,6,6-tetramethylpiperidine (TMP).

12. The method of either one of claim 10 or 11, wherein the deprotonating and oxidizing is performed in an organic solvent.

13. The method of claim 12, wherein the organic solvent is tetrahydrofuran (THF).

14. The method of any one of claims 10-13, wherein the deprotonating and oxidizing is performed at least in part at a temperature below 70° C.

15. The method of any one of claims 10-14, wherein the deprotonation occurs at a regioselectivity of approximately 3:1 or greater.

16. The method of any one of claims 10-15, wherein the oxidizing further comprises a borylation.

17. The method of any one of claims 10-16, wherein the one or more reagents comprise B(OiPr)₃ and an oxidizing agent.

18. The method of claim 17, wherein the oxidizing agent is hydrogen peroxide.

19. A method for the preparation of artemisinic acid comprising:
(a) deprotonating and oxidizing 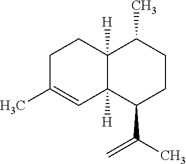
with one or more reagents under suitable conditions to make 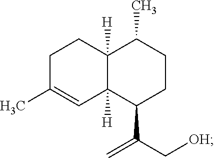 [SMILES: C=C(CO)[C@@H]1CC[C@@H](C)[C@@H]2CCC(C)=C[C@@H]21]
(b) oxidizing 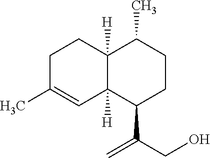 [SMILES: C=C(CO)[C@@H]1CC[C@@H](C)[C@@H]2CCC(C)=C[C@@H]21]
with one or more reagents under suitable conditions to make 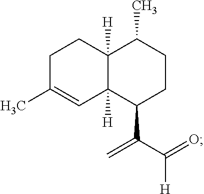 [SMILES: C=C(C=O)[C@@H]1CC[C@@H](C)[C@@H]2CCC(C)=C[C@@H]21]
(c) oxidizing 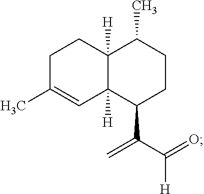 [SMILES: C=C(C=O)[C@@H]1CC[C@@H](C)[C@@H]2CCC(C)=C[C@@H]21]
with one or more reagents under suitable conditions to make artesimic acid.

20. The method of claim 19, wherein the one or more reagents of step (a) comprise n-BuLi, KOtBu, and 2,2,6,6-tetramethylpiperidine (TMP).

21. The method of either one of claims 19-20, wherein step (a) is performed in an organic solvent.

22. The method of claim 21, wherein the organic solvent is tetrahydrofuran (THF).

23. The method of any one of claims 19-22, wherein step (a) is performed at least in part at a temperature below 70° C.

24. The method of any one of claims 19-23, wherein the deprotonation of step (a) occurs at a regioselectivity of approximately 3:1 or greater.

25. The method of any one of claims 19-24, wherein the step (a) further comprises a borylation.

26. The method of any one of claims 19-25, wherein the one or more reagents of step (a) comprise B(OiPr)₃ and an oxidizing agent.

27. The method of claim 26, wherein the oxidizing agent is hydrogen peroxide.

28. The method of any of claims 19-27, wherein the one or more reagents of step (b) comprise 02.

29. The method of any of claims 19-28, wherein the oxidation of step (b) occurs in the presence of a Cu-catalyst.

30. The method of claim 29, wherein the Cu-catalyst is CuBr.

31. The method of any of claims 19-30, wherein step (b) is performed in an organic solvent.

32. The method of claim 31, wherein the organic solvent is acetonitrile (ACN).

33. The method of any of claims 19-32, wherein the oxidation of step (c) is a Pinnick oxidation.

34. The method of any of claims 19-33, wherein the one or more reagents of step (c) comprise 2-methyl-2-butene.

35. A method of regioselectively deprotonating and oxidizing an exocyclic carbon-carbon double bond over an endocyclic carbon-carbon double, comprising contacting a substrate having both an exocyclic carbon-carbon double bond and an endocyclic carbon-carbon double bond with one or more reagents under suitable conditions to convert the substrate's exocyclic carbon-carbon double bond into a carbon-carbon single bond with a hydroxy substituent.

36. The method of claim 35, wherein the one or more reagents comprise n-BuLi, KOtBu, and 2,2,6,6-tetramethylpiperidine (TMP).

37. The method of either one of claim 35 or 36, wherein the deprotonating and oxidizing is performed in an organic solvent.

38. The method of claim 37, wherein the organic solvent is tetrahydrofuran (THF).

39. The method of any one of claims 35-38, wherein the deprotonating and oxidizing is performed at least in part at a temperature below 70° C.

40. The method of any one of claims 35-39, wherein the deprotonation occurs at a regioselectivity of approximately 3:1 or greater.

41. The method of any one of claims 35-40, wherein the oxidizing further comprises a borylation.

42. The method of any one of claims 35-41, wherein the one or more reagents comprise B(OiPr)₃ and an oxidizing agent.

43. The method of claim 42, wherein the oxidizing agent is hydrogen peroxide.
